(19) United States
(12) Patent Application Publication
(10) Pub. No.: US 2026/0273156 A1
(43) Pub. Date: Sep. 17, 2026

(54) SYSTEM FOR DRAINAGE

(71) Applicant: SmartCanula LLC, Lausanne (CH)

(72) Inventors: Ludwig Karl Von Segesser, Stansstad (CH); Andreas Terpin, Lausanne (CH)

(21) Appl. No.: 19/469,228

(22) PCT Filed: Mar. 28, 2024
(86) PCT No.: PCT/EP2024/000020

§ 371 Date: Sep. 25, 2025

(30) Foreign Application Priority Data
Mar. 31, 2023 (EP) 23166004.4

(51) Int. Cl. A61M 1/36; A61M 39/00; A61M 39/10

(52) U.S. Cl. CPC A61M 1/3659; A61M 39/10; A61M 2039/0009; A61M 2039/1027; A61M 2039/1038

(57) ABSTRACT

System for vascular draining comprising a cannula for use in medical applications, wherein the cannula comprises a plurality of flexible filaments, such that the cannula can be varied between a normal and a low-profile configuration, wherein the proximal cannula portion is formed by a sleeve, and a plug device comprising a first and a second section, wherein the second section is configured to be releasable positioned into the sleeve, wherein the plug device comprises at least one fastening member configured to establish a press fit and/or a twist and/or a clamp connection between the sleeve and the plug device, such that when the plug device engages the sleeve, the cannula is transferred from the normal to the low-profile configuration.

### PRIOR ART

Cannulation is essential for extra-corporeal circulation in order to drain blood towards the life support system prior to reinjection into the circulation. For high flow applications like cardio-pulmonary bypass, extra-corporeal membrane oxygenation etc., performance of a cannula can be very important, because it is usually the narrowest part in the perfusion circuit. Conventional cannula designs are typically based on rectilinear designs, i.e., straight tubes. Thus, the resistance of such cannulas is increasing with cannula length in linear fashion. Hence, shorter cannulas can offer better performance. However, with venous cannulas, the tip of the cannula has to be positioned in the right atrium in order to avoid cannula orifice obstruction, thereby creating additional complications.

As a result, two approaches have been developed to improve venous drainage. One approach relates to making the cannula wall thinner in order to get a larger cross-sectional area and thus, providing less resistance. Another approach involves use of augmented venous drainage accomplished through a centrifugal pump or vacuum. However, because the latter approach requires an increased suction it likely results in cannula orifice obstruction and shut off of the venous drainage. This phenomenon is a typical finding in clinical cases undergoing minimal-invasive heart surgery with remote cannulation.

To overcome this problem, the EP 1 248 571 B1 or the EP 2 341 850 B1 describe a cannula for vascular draining that has a small diameter only for insertion into a vessel. The cannula comprises a cannula body with a lumen extending between the proximal and distal ends of the cannula, wherein the cannula is made of a flexible material, and has at least one mechanism that upon actuation serves to alter the conformation of the cannula between a normal profile conformation in which the cannula has a certain, large lumen diameter for vascular draining, and a low profile conformation in which the lumen diameter of the cannula has been decreased for insertion of the cannula into the vessel of a patient. The cannula therefore comprises a watertight coating at its proximal end, e.g. the flexible material such as a series of interlaced or interwoven wires are coated at the proximal end, whereas at a distal end of the cannula, the blood flow occurs through the uncoated interlaced wires. After proper positioning of the cannula in a vessel, the distal end of the cannula is expanded from the low-profile configuration up to the surface of the interior wall of the vessel for draining, i.e. into the normal profile configuration.

To convert the cannula to a low-profile configuration for insertion into the patient's vessel, i.e., to stretch the flexible wire mesh of the cannula body, a mandrel can be inserted into the lumen of the cannula, i.e. from the proximal end of the cannula up to the distal end of the cannula and over a guidewire. The mandrel comprises an elongated member and a plug. The elongated member comprises a distal and a proximal end. The plug is positioned at the proximal end of the mandrel, i.e. the plug forms the proximal end of the mandrel. When inserted into the lumen of the cannula, the elongated member abuts with its distal end the tip of the cannula. At its proximal end, the plug that can be inserted into the proximal end of the cannula, whereas the proximal end of the cannula is formed by a sleeve. Due to the different lengths of the cannula and the mandrel, when a force is applied to the plug, e.g. the plug is pressed further into the opening of the cannula, i.e further into the sleeve, the cannula is tensioned. This force applied to the plug, tensions the wire mesh and transfers the cannula to the desired low-profile configuration for insertion into a hollow organ. In the low-profile configuration, the cannula comprises a certain diameter, i.e. the lumen of the cannula has a diameter, which is smaller than in the normal-profile configuration, where the lumen has a larger diameter.

The wire mesh is attached to a component at the distal end of the cannula and to the sleeve at the proximal end of the cannula. Therefore, the sleeve essentially forms the proximal end of the cannula.

At the beginning of a drainage and as long as the mandrel is still stretching the cannula, i.e. the cannula is in the low-profile configuration, liquid, e.g. blood from the patient, cannot penetrate into the interior of the cannula, since the large openings formed by the wire mesh inside the cannula are blocked by the surface of the elongated member of the mandrel. Therefore, the plug has to remain in its fastening position during insertion. To transfer the cannula to its normal-profile configuration, i.e. when fluid or blood flows through the openings in the cannula, the plug has to be removed from its fastening position, preferably in a stepwise manner.

In the patent application EP 2 341 850 B1 the plug therefore snugly fits into the proximal cannula portion, i.e. into the sleeve or alternatively a locking mechanism is described in FIGS. 12A-12C of EP 2 341 850 B1. To establish the snug fit or press fit connection between the plug and the sleeve, the plug is slightly tapered or conically shaped and the connection is maintained by the frictional forces. These forces appear when the inner diameter of the sleeve is slightly larger than the outer dimension of the plug section, which fits into the sleeve. Preferably, to increase the friction, the sleeve is made of an elastic material, such as silicon. The snug fit type of connection has the disadvantage, that due to some liquid, e.g. from cleaning of the mandrel prior to use, the frictional forces are reduced and the connection may loosen uncontrolled. Further, temperature may affect the elasticity of the material of the cannula sleeve, which impacts on the frictional forces and may additionally lead to an uncontrolled loosening of the connection.

The alternative locking mechanism described in EP 2 341 850 B1, uses a connector, which is inserted into the proximal cannula portion, and which comprises several re-hooks, which are configured to engage with a clamp. The clamp comprises the elongated member and therefore, engaging the clamp with one of the several re-hooks, tensions the cannula. The locking mechanism has the disadvantage that it additionally requires a connector. And further, it has to be monitored during production that the connector is connected to the proximal cannula portion in a way that it can withstand the mechanical traction necessary for tensioning of the cannula. Therefore, the described locking mechanism requires more expenses.

It is therefore an object of the invention to provide alternative solutions to the problem of releasable positioning of the plug in the proximal cannula portion, in order to reversibly transfer the cannula from its normal into its low-profile configuration and which overcome the aforementioned disadvantages. In particular, it should be possible to tension and release the cannula gradually, while keeping it tight against the loss of blood.

### GENERAL DESCRIPTION

The problem is solved by the subject matter of the independent claims, i.e. by a system for vascular drainage according to claim 1, a plug device according to claim 11, a support sleeve according to claim 12, a cannula according to claim 14 and a kit of parts according to claim 15. Further preferred embodiments of the invention are subject of the dependent claims.

The vascular drainage system according to the invention comprises a cannula, in particular for use in medical applications, with a distal cannula portion and a proximal cannula portion, wherein the cannula comprises a plurality of flexible filaments, such that the cannula can be varied between a normal and a low-profile configuration, wherein the proximal cannula portion is formed by a sleeve, and
a plug device comprising a first and a second section, wherein the second section is configured to be releasable positioned into the sleeve and the second section comprises an opening to receive a proximal portion of an elongated member and
wherein the plug device comprises at least one fastening member configured to establish a press fit and/or a twist and/or a clamp connection between the sleeve and the plug device, such that when the plug device engages the sleeve, the cannula is transferred from the normal to the low-profile configuration.

A drainage is a medical treatment method. It is used to drain or aspirate pathological or increased body fluids or gases in order to restore a normal condition. In principle, a distinction is made between internal and external drainage. In internal drainage, obstacles are surgically, e.g. minimally invasive, bypassed, or accumulated fluids are drained into hollow organs, e.g. the stomach or the intestines. In external drainage, the drainage is from the inside of the body to the outside.

A vascular drainage system, for example according to the “Seldinger-Method” may typically comprise a guide wire, inserted into the patient's vessel prior to draining and which serves to guide a cannula to a hollow organ, wherein the cannula is in principle, a flexible hollow tube through which the fluid to be drained is led to the outside of the vessel, i.e. out of the patient.

The cannula is designed to be inserted into a hollow organ, which can be selected from, for example, a vein, an artery, a urethra, a ureter, an intestine, an esophagus, a trachea, a bronchial tube, a pleural space, and/or a peritoneum.

Use of the cannula in medical contexts includes methods such as placing the cannula in its low-profile conformation, inserting the cannula into a hollow organ of a patient at a point of insertion, and returning the cannula to its normal-profile conformation. In the normal profile conformation, the cannula expands, in particular at its distal end, i.e. distal to the point of insertion, up to the diameter of the hollow organ or up to the maximum diameter of the cannula body, i.e. the lumen of the cannula, which is formed by the cannula body.

In the low-profile configuration of the cannula, the diameter of the cannula is reduced for insertion of the cannula into a hollow organ, for example into a vessel compared to the normal-profile configuration. In the low-profile configuration the wire mesh, i.e. the flexible filaments of the cannula form the cannula body, thereby defining a cannula lumen. In the low-profile configuration the flexible filaments are essentially tensioned compared to the normal-profile configuration. The diameter of the cannula refers to the diameter of the lumen, i.e. the inner diameter of the cannula and/or to the diameter of the cannula body, i.e. the outer diameter of the cannula. The diameter relates to the plurality of diameters which appear in the normal-profile configuration by the actual shape of the cannula body and/or lumen, but which alter by tensioning of the cannula when brought into the low-profile configuration.

When the cannula is in its normal profile conformation when in use, the lumen diameter distal to the point of insertion varies in relation to the diameter of the surrounding vessel. Further, the cannula is in its normal profile conformation when in use, the portion of the cannula distal to the point of insertion supports an inner surface of the surrounding vessel. When the cannula is in its low-profile configuration, a portion of the cannula is characterized by a narrow diameter of the lumen of the cannula that is suitable for insertion into the object to be cannulated as well as into smaller access vessels. However, placing the cannula in the low-profile configuration can be done before, during or after cannulation.

In order to allow for this variation of the cannula diameter, the cannula comprises flexible filaments, i.e. an elastic body formed at least partially from the flexible filaments. The plurality of flexible filaments may include one or more materials selected from metals, shape-memory metals, alloys, plastics, textile fibers, synthetic fibers, and/or combinations thereof. For example, the metal can be stainless steel. Moreover, the plurality of flexible filaments can have a shape selected from round, oval, flattened, triangular, rectangular and combinations thereof. In one embodiment, the plurality of flexible filaments are textile fibers. The plurality of flexible filaments can be braided together, knitted together, or interwoven. Alternatively, the plurality of flexible filaments is interlaced.

The elastic filaments enclose a volume, which can be defined as the inner lumen of the cannula and whose spatial extent can be varied due to the elastic filaments. The lumen has a distal end pointing towards the insertion point and a proximal end, pointing in the opposite direction away from the insertion point, i.e., for example, in a direction away from the patient. Consequently, the cannula comprises a distal cannula portion, which is the portion close to the insertion point and a proximal cannula portion, which is the portion further away from the insertion point.

To change the spatial extent of the lumen of the cannula, i.e. to tension the cannula by conformation from the normal into the low-profile configuration, a mandrel is inserted into the lumen from the proximal cannula portion to the distal cannula portion. The mandrel comprises an elongated member and a plug. The elongated member has a distal end and a proximal end, wherein the plug is positioned at the proximal end of the elongated member. To allow the diameter of the cannula, or the lumen of the cannula, to vary between the low-profile configuration and the normal profile configuration, the mandrel, i.e. the plug with the elongated member connected to it, is inserted into the lumen of the cannula, such that the distal end of the elongated member engages the distal portion of the cannula. The plug is then pushed further into the lumen of the cannula, i.e. into the proximal portion of the cannula. Thereby a force is created that tensions the flexible filaments of the cannula and thus changes the diameter of the cannula from the normal-profile configuration to the low-profile configuration. Upon removal of the mandrel, the cannula will expand to its unclamped diameter, in particular by itself, due to the tendency of the flexible filaments to assume their original normal shape, like a spring, whereas the normal shape relates to a larger diameter of the lumen at the point of insertion. The elongated member of the mandrel may, in particular for medical applications, comprise a hollow tube and may be configured for insertion of a guide wire, through the hollow tube. In this case, the guide wire passes through the elongated member up to the distal end of the cannula and out through the distal end of the cannula into the hollow organ, e.g. a vein, in order to further guide the cannula to the application site through the vessel, e.g. the vein. The guide wire can then be inserted into and removed from the hollow organ via the mandrel positioned inside the body of the cannula. Further, the mandrel can be removed from the cannula whereas the guide wire remains positioned inside the cannula and the hollow organ.

The elongated member preferably comprises the hollow tube and the plug of the mandrel may therefore comprise an opening configured for insertion of the guide wire. The plug is positioned at the proximal end of the mandrel and the elongated member connects the plug with the distal end of the elongated member, thereby forming the mandrel.

The cannula comprises a distal cannula portion and a proximal cannula portion, wherein the proximal cannula portion is formed by a sleeve. A sleeve refers to, for example a tubing, in particular an elastic tubing, e.g. made of silicon, which serves to attach the flexible filaments to the proximal end of the cannula, such that a certain lumen diameter of the cannula is permanently present at the proximal end of the cannula and allows to connect the cannula to, for example, a cardiovascular machine.

A plug device refers to a device, which is configured to be partially inserted into the proximal cannula portion, i.e. the sleeve or the support sleeve. The plug device in principle is similar to a cork that is partially pressed into the neck of a bottle to seal it from the outside. Therefore, the plug comprises a first and a second section, wherein the second section corresponds to the part, which is releasably inserted into the sleeve. The second section is inserted into the sleeve for tensioning of the cannula and released from the sleeve to untension the cannula. The first section may serve to provide an application surface, where the plug device can be pulled out of the sleeve. The first section also mechanically supports the second section. The plug comprises at least one fastening element, which can be positioned on the first and/or the second section. The plug device preferably further comprises an opening, such that a guide wire can be inserted through the plug.

The plug comprises at least one fastening member. The fastening member is configured to establish a press fit and/or a twist and/or a clamp connection between the sleeve and the plug device, such that when the plug device engages the sleeve, the cannula is transferred from the normal to the low-profile configuration. Therefore, a fastening member relates to, in particular a member that can provide a press fit and/or rotary and/or clamping connection between the plug and the sleeve or the support sleeve. Such a member can be for example a protrusion, which, for example is positioned on the surface of the second section. The surface of the second section is exposed to the inner surface of the sleeve or the support sleeve, such that adjacent surfaces are obtained. The mutual contact pressure of the adjacent surfaces generates, for example a press fit connection, whereas in such a case the protrusions support the press fitting by increasing the frictional force, i.e. the mutual contact pressure, between the inner surface of the sleeve or the support sleeve and the surface of the second section. The protrusions can take a variety of shapes as long as they increase in particular the frictional force, i.e. the mutual contact pressure, of the adjacent surfaces.

A further example of a fastening member is a thread or a slot. By means of a thread a rotary, i.e. a twist connection can be made between the plug and the sleeve or the support sleeve. Such a rotary connection has the advantage that a plurality of different forces for tensioning of the cannula can be adjusted. Therefore, the transition from the low-to the normal profile configuration can be executed in a very dosed manner, depending on the pitch of the thread.

Another example of a fastening member is a slot, which is configured to receive a protrusion, such that the protrusions can be retained inside the slot when the cannula is in the low-profile configuration. The plug may comprise a plurality of slots to realize different holding positions, in order to stepwise tension or release the cannula.

Therefore, examples of fastening members can be a thread or a ring or a slot or pluralities thereof or any combinations thereof. One fastening member may also comprise combinations of, for example a thread and/or a protrusion and/or a slot or any combination of pluralities thereof.

A press fit connection hereby relates to a connection type which, for example is based on connection pressure between two neighboring surfaces. Therefore, when the two neighboring surfaces, i.e. mating parts, are pressing against each other the, in particular frictional and or adhesive forces, are increased, such that the two surfaces no longer move relative to each other. Accordingly, a press fit, also known as an interference fit, or a friction fit is a form of fastening between the two tight fitting mating parts that produces a joint which is held together by friction after the parts are pushed together. The tightness of fit is controlled by amount of interference, that is the allowance, i.e. the planned difference from nominal size. In the present case, this could be the tapering angle β of the second section of the plug and/or the size of the protrusion and/or the inner diameter of the sleeve or the support sleeve into which the second section is inserted.

A twist connection hereby relates to a connection type, which is essentially based on a rotary joint or a rotating connection or a rotary union, as for example a connection by means of two interlocking threads, or a bayonet fastener, or any type of connection between two components, e.g. the sleeve and the plug, that requires a rotational movement to close and/or open the connection.

A clamp connection hereby relates to a connection type, which is based on a clamping between to components, e.g. between the fastening member and the protrusions. Clamp connections are usually detachable connections between two components. The frictional connection is created by means of screws or springs pretensioned during assembly. For example, a clamping connection may be provided by a pincer-shaped clamp, such as a clothespin for clamping laundry.

The invention also relates to a plug for use in a, in particular medical, cannula, wherein the cannula comprises a proximal cannula portion formed by a sleeve, in particular by a support sleeve, and a plurality of flexible filaments, such that the cannula can be varied between a normal and a low-profile configuration, the plug device comprising a first and a second section, wherein the second section is configured to be releasable positioned into the sleeve, in particular into the support sleeve, and comprises an opening to receive a proximal portion of an elongated member and wherein the plug device comprises at least one fastening member configured to establish a press fit and/or a twist and/or a clamp connection between the sleeve, in particular between the support sleeve, and the plug device, such that when the plug device engages the sleeve, in particular the support sleeve, the cannula is transferred from the normal to the low-profile configuration.

The invention also relates to support sleeve, for use in a cannula, in particular for medical applications, to form a proximal cannula portion, wherein the cannula comprises a plurality of flexible filaments, such that the cannula can be varied between a normal and a low-profile configuration, wherein the support sleeve comprises a, in particular external, thread and/or a protrusion and/or a slot configured to establish a press fit and/or a twist and/or a clamp connection between the cannula and a plug device. The cannula in particular further comprises a sleeve, which forms the proximal cannula portion, such that the support sleeve can be slid and positioned over the sleeve, thereby forming the proximal cannula portion.

The invention also relates to a cannula, in particular for use in medical applications, with a distal cannula portion and a proximal cannula portion, wherein the cannula comprises a plurality of flexible filaments, such that the cannula can be varied between a normal and a low-profile configuration, wherein the proximal cannula portion is formed by a sleeve, in particular by a support sleeve, wherein the sleeve, in particular the support sleeve, comprises a, in particular external, thread and/or a protrusion and/or a slot configured to establish a press fit and/or a twist and/or a clamp connection between the sleeve, in particular between the support sleeve, and a plug device.

The invention also relates to a kit of parts comprising, a cannula, in particular for use in medical applications, with a distal cannula portion and a proximal cannula portion, wherein the cannula comprises a plurality of flexible filaments, such that the cannula can be varied between a normal and a low-profile configuration, wherein the proximal cannula portion is formed by a sleeve, in particular by a support sleeve, and a plug device comprising a first and a second section, wherein the second section is configured to be releasable positioned into the sleeve, in particular into the support sleeve, and comprises an opening to receive a proximal portion of an elongated member and wherein the plug device comprises at least one fastening member configured to establish a press fit and/or a twist and/or a clamp connection between the sleeve, in particular between the support sleeve, and the plug device, such that when the plug device engages the sleeve, the cannula is transferred from the normal to the low-profile configuration.

In a preferred embodiment of the system and of the cannula the proximal cannula portion is formed by a support sleeve. The support sleeve refers to an additional sleeve, which is at least partially positioned over the sleeve at the proximal cannula portion and thus forms the proximal cannula portion instead of the sleeve, but however has essentially the same effect. Use of a support sleeve has the advantage, that the cannula can be manufactured without finalizing the type of connection mechanism between the proximal cannula portion and the plug. This allows manufacturing costs to be reduced, as the support sleeve can be pre-produced in different versions, in large quantities. The different versions of the support sleeve comprise those of, for example threads, protrusions and/or slots, to establish a press fit and/or a twist and/or a clamp connection between the cannula and the plug device.

In a preferred embodiment of the system and the plug, in particular for the press fit connection, the fastening member comprises a, in particular ring-shaped or an annular, protrusion. In this preferred embodiment, the second section, which is inserted into the sleeve and or into the support sleeve, comprises a ring-shaped or annular protrusion or a plurality thereof. This allows the contact pressure between the plug and the sleeve to be advantageously increased. Preferably, the ring-shaped or annular protrusions are arranged next to each other in such a way as to enable the plug to be released in stages; if, for example, there are three ring-shaped or annular protrusions, the plug is pulled out to such an extent that the other two protrusions remain in the sleeve and the contact pressure is sufficiently high that the plug does not pop out in an uncontrolled manner.

In a preferred embodiment of the system, the plug and the cannula, in particular for the twist connection, the sleeve comprises a, in particular external, thread and the fastening member comprises a, in particular an internal, thread, configured to rotatably engage with each other. In such an embodiment tensioning of the cannula can be stepless therefore, giving the user maximum control over the desired profile configuration of the cannula. In the particular case where the fastening member comprises an internal thread, the thread is positioned on the first section of the plug, such that the inside of the cannula retains its smooth surface to prevent contamination.

In a preferred embodiment of the system, the plug and the cannula, in particular for the twist connection, the sleeve comprises a protrusion and the fastening member comprises a slot, wherein the slot is configured to accommodate the protrusion. This embodiment relates to a twist connection, i.e. a rotary coupling between the plug and the proximal cannula portion. Preferably the fastening member comprises a plurality of slots, arranged such that changing the protrusion from one slot position to a further slot position corresponds to an increase in the tension of the cannula. Therefore, the user gains control over the desired profile configuration gradually.

In a particular preferred embodiment of the system, the plug and the cannula, the slot is “L” or “T” shaped, such that the protrusion is first inserted into the slot and then anchored there by a rotary movement.

In a particular preferred embodiment of the system, the plug and the cannula, in particular for the clamp connection, the sleeve comprises at least one protrusion and the plug device comprises at least one clamp member, wherein the clamp member comprises the fastening member, which is configured to engage with the at least one protrusion of the sleeve or the support sleeve. The fastening member refers to, for example a protrusion. The connection is established when the fastening member hooks into the protrusion. Therefore, the fastening member and the protrusion can, for example, be shaped complementary to each other to facilitate the interlocking of both elements. To engage or disengage the connection, the fastening member is released from the connection with the protrusion by actuating the clamp member. This can be achieved, for example, by a pincer-shaped design of the clamp member, whereby the fastening member is located at the tip of the pincer. The embodiment advantageously provides the user good control over the desired profile configuration of the cannula gradually, without the risk that the plug device accidentally releases itself due to a decrease in the frictional forces.

In a particular preferred embodiment of the system and the cannula, the sleeve comprises a beveled proximal portion. This creates a gap between the first section and the second section, when the second section is inserted into the sleeve, and it is therefore easier for the user to reach into manually to remove the plug from the sleeve.

In a particular preferred embodiment of the system and the plug, the second section of the plug device is at least partially tapered, in particular at least partially conically shaped. By means of a tapered or conical second section of the plug device, the contact pressure between the plug device and the sleeve can be preset. This means that, depending on the angle of inclination, a larger or a smaller surface area of the second section is pressed against the inner wall of the sleeve. In combination with, for example ring shaped or annular protrusions, the contact pressure can be preset by the angle of inclination and the height of the protrusions positioned on the second section.

In a particular preferred embodiment of the system and the plug, the first and the second section are formed cylindrically and wherein the second section has a smaller diameter than the first section. The different diameters simplify manual extraction of the plug from the sleeve by the user.

In a preferred embodiment of the support sleeve, the support sleeve comprises a beveled proximal portion. This creates a gap between the first section and the second section of the plug, when the second section is inserted into the support sleeve, and it is therefore easier for the user to reach into manually to remove the plug from the sleeve.

### BRIEF DESCRIPTION OF THE FIGURES

FIG. 1 Illustrates a cross-sectional view of the system according to one embodiment, comprising a cannula and a mandrel, wherein the cannula is in a low-profile configuration and wherein the plug is fully inserted into the lumen of the cannula and comprises four ring-shaped protrusions.

FIG. 2 Illustrates a cross-sectional view of the system according to one embodiment, comprising a cannula and a mandrel, in a normal profile configuration, wherein the plug is partially conically shaped and comprises three ring-shaped protrusions.

FIG. 3 Illustrates a zoom view of the proximal end of the system according to one embodiment, wherein the plug is partially conically shaped and comprises three ring-shaped protrusions. The sleeve comprises a bevelled proximal portion to form a gap.

FIG. 4 Illustrates three cross-sectional views of the system according to one embodiment, wherein the fastening member comprises an internal thread and the sleeve comprises an external thread.

FIG. 5 Illustrates two cross-sectional views of the system according to one embodiment, wherein the fastening member comprises an internal thread and the support sleeve comprises an external thread.

FIG. 6 Illustrates a cross-sectional view of the system according to one embodiment, wherein the fastening member comprises three “L”-shaped slots and the sleeve comprises a protrusion.

FIG. 7 Illustrates a cross-sectional view of the system according to one embodiment, wherein the fastening member comprises three “L”-shaped slots and the support sleeve comprises a protrusion.

FIG. 8 Illustrates a cross-sectional view of the system according to one embodiment, comprising a clamp connection. The sleeve comprises several protrusions, which engage with the fastening members positioned at the tip of a pincer-shaped clamp member.

FIG. 9 Illustrates a cross-sectional view of the system according to one embodiment, comprising a clamp connection. The support sleeve comprises several protrusions, which engage with fastening members positioned at the tip of a pincer-shaped clamp member.

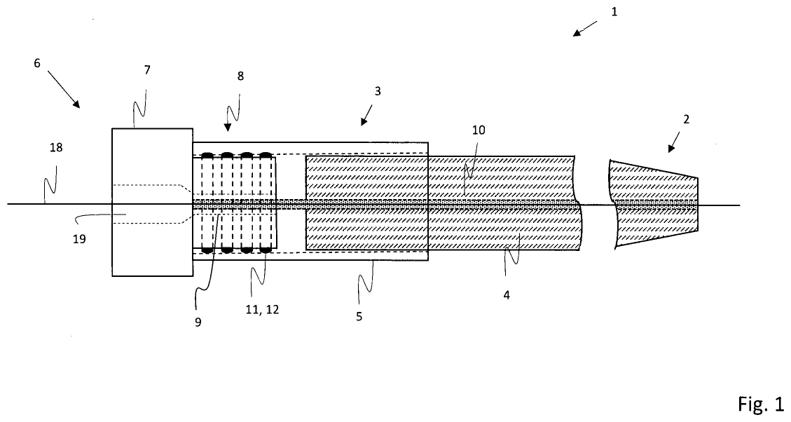

In FIG. 1 a cross-sectional view of the system according to one embodiment is shown, comprising a cannula 1 and a mandrel, wherein the cannula 1 is in a normal configuration and wherein the mandrel is partially inserted into the lumen of the cannula body. The cannula body comprises a distal cannula portion 2 and a proximal cannula portion 3, wherein the proximal cannula portion 3 comprises a sleeve 3 to which the plurality of flexible filaments 4, which form the flexible body portion of the cannula 1, are attached. The mandrel comprises a plug 6 and an elongated member 10, with a distal and a proximal end, wherein the plug 6 connects to the proximal end of the elongated member 10. With its second section 8, the plug snug fits into the sleeve 3 of the cannula 1, and which forms the proximal cannula portion 3, thereby sealing the inside of the proximal cannula portion 3 in a fluid tight manner. The first section 7 of the plug remains outside the proximal cannula portion 3. The second section 8 is defined as the part of the plug 6, which is inserted into the sleeve 5. The plug 6 comprises an opening 9, into which the proximal portion of the elongated member 10 is inserted and to which it is fixed. The plug 6 comprises four ring shaped protrusions 12 as fastening members 11, which serve to increase the frictional force between the inside surface of the sleeve 5 and the outside surface of the second section 8 of the plug 6 to establish a press fit connection between the sleeve 5 and the plug 6. Further a guide wire 18 is inserted into the plug 6 through a guide wire insertion opening 19 of the plug 6.

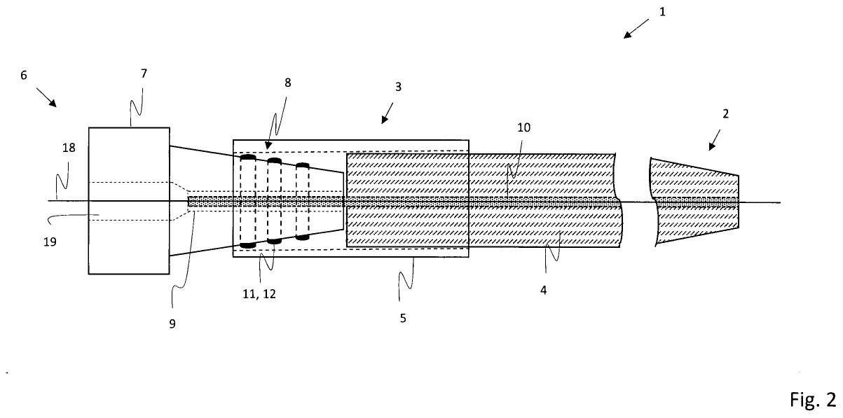

FIG. 2 illustrates a cross-sectional view of the system according to one embodiment, comprising a cannula 1 and a mandrel, in a normal profile configuration, wherein the plug 6 is partially conically shaped and comprises 3 ring-shaped protrusions 12 as fastening members 11. The ring-shaped protrusions 12 are positioned on the tapered surface of the second section 8 of the plug 6. The tapering angle defines in combination with the dimension of the ring-shaped protrusions 12 the contact pressure between the second section 8 of the plug and the sleeve 5. The ring-shaped protrusions 12 are arranged side by side, such that when the plug 6 is pulled back from the sleeve 5 to transfer the cannula 1 from the low-profile into the normal profile configuration, the contact pressure is stepwise reduced, such that the plug 6 does not accidentally loosens completely.

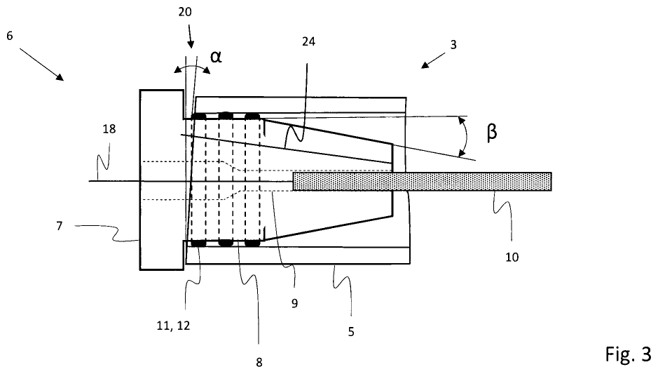

FIG. 3 Illustrates a zoom view of the proximal end of the system according to one embodiment, wherein the plug 6 is partially conically shaped and comprises three ring-shaped protrusions 12. The sleeve 5 comprises a bevelled proximal portion, which creates a gap 20 between the first section 7 and the second section 8 of the plug 6. When the second section 8 is inserted into the sleeve 5, it is therefore easier for the user to reach into manually, to remove the plug 6 from the sleeve 5. The gap depends on the bevel angle a, i.e. the bevel angle a defines the gap. In the embodiment shown in FIG. 3, the second section 8 comprises three ring-shaped or annular protrusions 12 in the non-tapered region, whereas the further region of the second section 8 is tapered. The taper angle is defined by β and can be adjusted, such that the connection pressure is a combination of the press fitting through the ring-shaped or annular protrusions 12 and the press fitting through the tapering. Therefore, when the plug 6 is removed from the sleeve 5, the press fitting due to the tapering still creates some weaker connection pressure to avoid that the plug 6 fully loosens at once. This additionally facilitates operating the cannula in a controllable manner. The plug 6 further comprises a slit 24, which runs through the first 7 and second section of the plug 6. The slit 24 serves to allow fluid pressure compensation between the inside and the outside of the cannula 1.

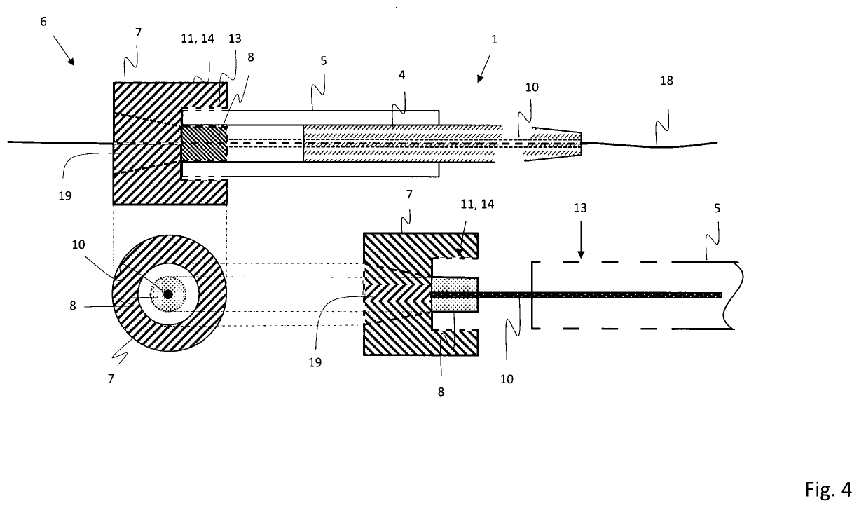

FIG. 4 illustrates cross-sectional views of the system according to one embodiment. The fastening member 11 comprises an internal thread 14 and the sleeve 5 comprises an external thread 13. Therefore, the plug 6 is U-shaped, whereas the second section 8 is symmetrically arranged like a pin in the center of the U-shaped plug 6. The sleeve 5 engages with its external thread 13 in the internal thread 14 of the plug 6, thereby centrally receiving the pin-shaped second section 8. The further the plug 6 is screwed onto the sleeve 5, the more the tension of the cannula 1 increases. Depending on the pitch of the threads 13, 14, this allows continuous adjustment of the tension of the cannula 1. The second section further comprises the opening 19 for insertion of a guidewire 18. The second section 8 comprises the elongated member 10. The entire embodiment is configured rotationally symmetrical. Alternative, not shown in the FIG. 4, the second section 8 could also have an external thread 14 and the sleeve 5 an internal thread 13. However, the embodiment shown in FIG. 4 advantageously reduces the risk of contamination because the sleeve 5 internally does comprise a flat inner surface, which is easy to clean.

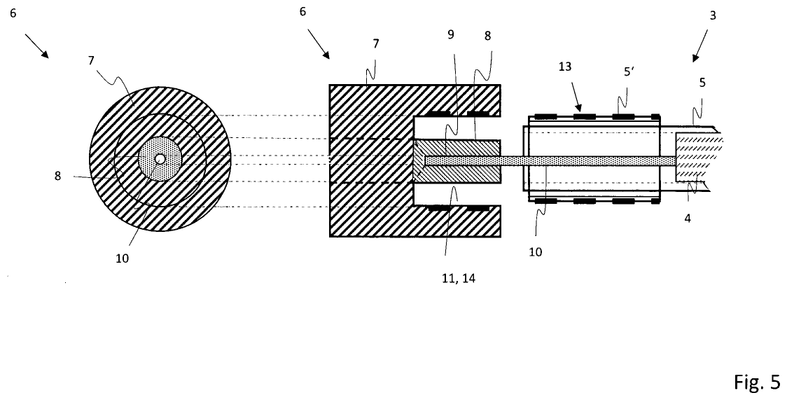

FIG. 5 illustrates two cross-sectional views of the system according to one embodiment, wherein the fastening member 11 comprises an internal thread 14 and the support sleeve 17 comprises an external thread 13. Basically, the illustration shown in FIG. 5 differs from that shown in FIG. 4 only in that the thread 13 is located in the support sleeve 17 and the support sleeve 17 is pushed onto the sleeve 5 and fastened. Therefore, the support sleeve 17 has essentially the same effect as the sleeve 5 shown in FIG. 4.

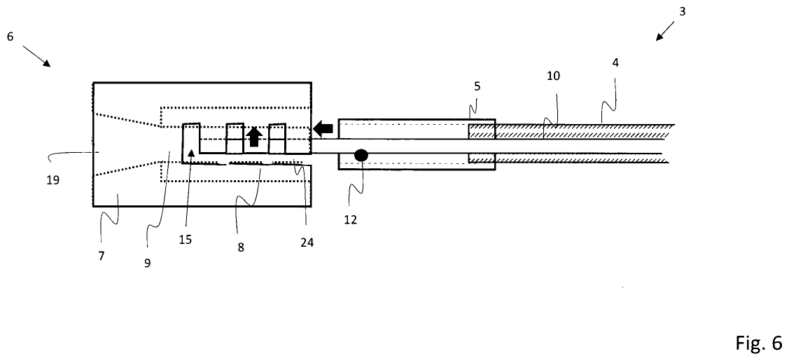

FIG. 6 illustrates a cross-sectional view of the system according to one embodiment, wherein the fastening member 11 comprises three L-shaped slots 15 and the sleeve 5 comprises a protrusion 12. To establish the connection between the plug 6 and the sleeve 5, the sleeve 5 is inserted centrally into the plug 6, as it is indicated by the arrow in FIG. 6. The protrusion 12 moves within the slit 24 provided for this purpose and which results from the “L” shaped geometry of the three slots 15, arranged, for example along a central symmetry axis of the embodiment. A rotation, which is indicated by another arrow in FIG. 6, engages the protrusion 12 of the sleeve 5 into the plug 6, such that the tension of the cannula 1 built up by the insertion of the sleeve 5 into the plug 6 is maintained. By further inserting the sleeve 5 into the next slot 15 of the plug 6, the tension of the cannula 1 can be increased. The tension levels of the cannula 1 can be graded by the number of slots 15. The distances between the slots 15 determine the level of tension of the cannula 1 as such. The slots 15 as shown in FIG. 6 are “L”-shaped. However, “T”-shaped slots 15 are possible as well or any other shape configured to receive and to hold a protrusion 12.

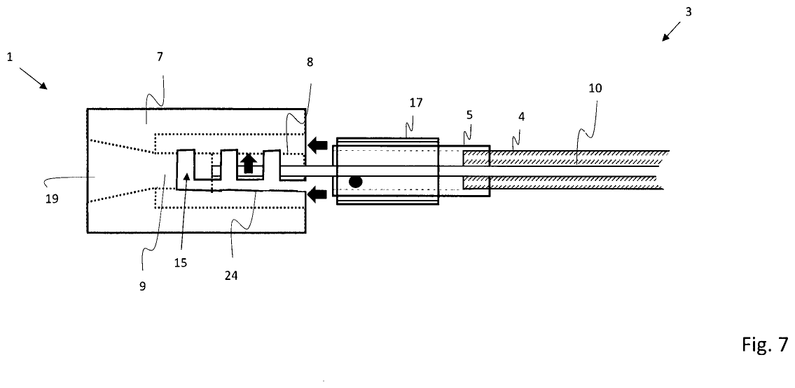

FIG. 7 illustrates a cross-sectional view of the system according to one embodiment, wherein the fastening member 11 comprises three “L”-shaped slots 15 and the support sleeve 17 comprises a protrusion 12. Therefore, the embodiment shown in FIG. 7 differs from the embodiment shown in FIG. 6 that the support sleeve 17 is pushed onto the sleeve 5, wherein the support sleeve 17 has essentially the same effect as the sleeve 5 as already described for FIG. 6.

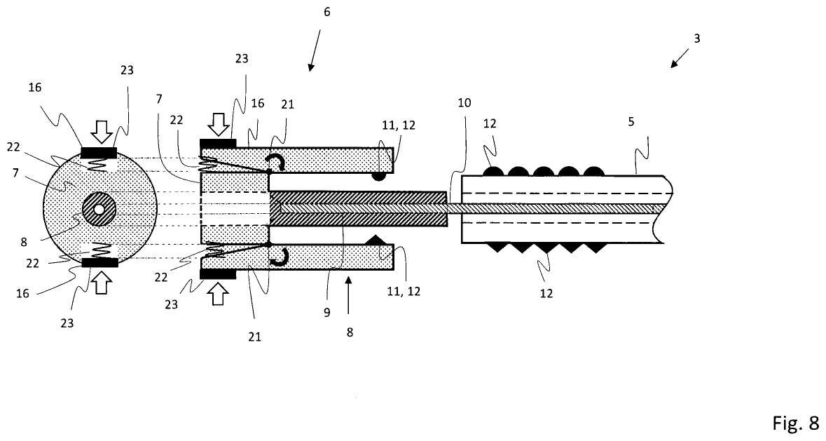

FIG. 8 illustrates a cross-sectional view of the system according to one embodiment, comprising a clamp connection. The sleeve 5 comprises several protrusions 12, which engage with the fastening members 11 positioned at the tip of a pincer-shaped clamp member 16. Therefore, the fastening members 11 can be protrusions 12, which are in particular shaped complementary to the protrusions 12 positioned on the sleeve 5, to facilitate clamping between the sleeve 5 and the plug 6. To establish the connection between the plug 6 and the sleeve 5, the sleeve 5 is pushed over the second section 8 towards the proximal portion of the plug 6, while the clamp member 16 is open. In FIG. 8 the clamp member 16 is shown in the closed position. The open position is achieved by pressing the control surface 23, in particular manually, so that the clamp member 16 is pivoted away from the sleeve 5 about an axis of rotation defined by a joint 21 for receiving the sleeve 5. If the control surface 23 is no longer actuated, then the clamp member 16 automatically swings back in the direction of the sleeve 5 due to the spring element 22 and a connection is created by the mutual engagement of the fastening member 11 with the protrusions 12 of the sleeve 5. The embodiment as shown in FIG. 8 has a rotationally symmetrical structure. The distance between the protrusions 12 on the sleeve 5 define variable clamp positions between the plug 6 and the sleeve 5, which corresponds to the different levels of tension of the cannula 1. The tension level is graded by the number of protrusions 12 arranged on the sleeve 5. The plug 6 further comprises an opening 9 configured to receive the proximal end of the elongated member 10. The spring element 22 can be a metal spring or some elastic material, which deforms upon actuation, for example. The clamp element 16 may be made of metal or plastic and may be attached to the first 7 or second 8 section by the joint 21, which may be a hinge, for example, thereby forming a part of the first section 7. The clamp member 16 is pincer-shaped, and the fastening members 11 are preferably located at the tip of the pincer. In the embodiment shown in FIG. 8, the plug 6 is rotationally symmetrical and has two opposing clamp members 16. Alternatively, fewer or more clamp members 16 may be arranged on the plug 6. The fastening members 11 at a respective clamp member 16 may have the same or different shape. The fastening elements 11 on a respective clamping element 16 are preferably adapted to their respective counterpart, namely the projections 12 located on the sleeve 5. For example, by complementary shaping to favour the engagement of fastening member 11 and protrusion 12.

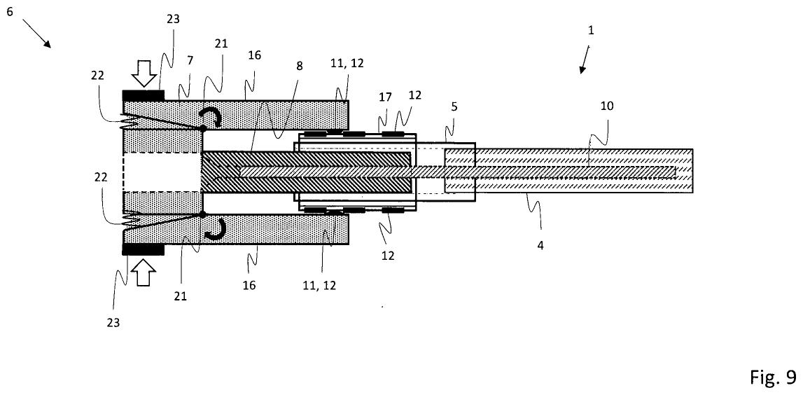

FIG. 9 illustrates a cross-sectional view of the system according to one further embodiment, comprising a clamp connection. In the embodiment shown in FIG. 9 a support sleeve 17 is additionally used and comprises several protrusions 12, which engage with fastening members 11 positioned at the tip of two pincer-shaped clamp members 16. The support sleeve 17 is pushed over the sleeve 5 and has essentially the same effect as the sleeve 5 as shown in FIG. 8. Therefore, for the embodiment shown in FIG. 9, the description given for FIG. 8 can essentially be referred to.

### LIST OF REFERENCE SIGNS

1 cannula
2 distal cannula portion
3 proximal cannula portion
4 flexible filaments
5 sleeve
6 plug device
7 first section of the plug device
8 second section of the plug device
9 opening of the plug device
10 elongated member
11 fastening member
12 protrusion
13 thread of sleeve
14 thread as fastening member
15 slot
16 clamp member
17 support sleeve
18 guidewire
19 guidewire insertion opening
20 gap
21 joint
22 spring element
23 control surface
24 slit

1. System for vascular draining comprising
a cannula (1), in particular for use in medical applications, with a distal cannula portion (2) and a proximal cannula portion (3), wherein the cannula (1) comprises a plurality of flexible filaments (4), such that the cannula (1) can be varied between a normal and a low-profile configuration, wherein the proximal cannula portion (3) is formed by a sleeve (5), and
a plug device (6) comprising a first (7) and a second (8) section, wherein the second section (8) is configured to be releasable positioned into the sleeve (5) and the second section comprises an opening (9) to receive a proximal portion of an elongated member (10) and
wherein the plug device (6) comprises at least one fastening member (11) configured to establish a press fit and/or a twist and/or a clamp connection between the sleeve (5) and the plug device (6), such that when the plug device (6) engages the sleeve (5), the cannula (1) is transferred from the normal to the low-profile configuration.

2. System according to claim 1, wherein the proximal cannula portion (3) is formed by a support sleeve (17).

3. System according to any of the previous claims, wherein, in particular for the press fit connection, the fastening member (11) comprises a, in particular ring-shaped, protrusion (12).

4. System according to any of the previous claims, wherein, in particular for the twist connection, the sleeve (5) comprises a, in particular external, thread (13) and the fastening member (11) comprises a, in particular internal, thread (14), configured to rotatably engage with each other.

5. System according to any of the previous claims, wherein, in particular for the twist connection, the sleeve (5) comprises a protrusion (12) and the fastening member (11) comprises a slot (15), wherein the slot (15) is configured to accommodate the protrusion (12).

6. System according to claim 5, wherein the slot is “L” or “T” shaped.

7. System according to any of the previous claims, wherein, in particular for the clamp connection, the sleeve 5 comprises a protrusion (12) and the plug device (6) comprises a clamp member (16), wherein the clamp member (16) comprises the fastening member (11), which is configured to engage with the protrusion (12) of the sleeve.

8. System according to any of the previous claims, wherein the sleeve (5) comprises a beveled proximal portion.

9. System according to any of the previous claims, wherein the second section (8) is at least partially tapered, in particular at least partially conically shaped.

10. System according to any of the previous claims, wherein the first (7) and the second (8) section are formed cylindrically and wherein the second section (8) has a smaller diameter than the first section (7).

11. Plug device for use in a, in particular medical, cannula, wherein the cannula (1) comprises a proximal cannula portion (3) formed by a sleeve (5), in particular by a support sleeve (17), and a plurality of flexible filaments (4), such that the cannula (1) can be varied between a normal and a low-profile configuration, the plug device (6) comprising
a first (7) and a second (8) section, wherein the second section (8) is configured to be releasable positioned into the sleeve (5), in particular into the support sleeve (17), and comprises an opening (9) to receive a proximal portion of an elongated member (10) and wherein the plug device (6) comprises at least one fastening member (11) configured to establish a press fit and/or a twist and/or a clamp connection between the sleeve (5), in particular between the support sleeve (17), and the plug device (6), such that when the plug device (6) engages the sleeve (5), in particular the support sleeve (17), the cannula (1) is transferred from the normal to the low-profile configuration.

12. Support sleeve, for use in a cannula (1), in particular for medical applications, to form a proximal cannula portion (3), wherein the cannula (1) comprises a plurality of flexible filaments (4), such that the cannula (1) can be varied between a normal and a low-profile configuration, wherein the support sleeve (17) comprises a, in particular external, thread (13) and/or a protrusion (12) and/or a slot (15) configured to establish a press fit and/or a twist and/or a clamp connection between the cannula (1) and a plug device (6).

13. Support sleeve according to claim 12, comprising a beveled proximal portion.

14. Cannula, in particular for use in medical applications, with a distal cannula portion (2) and a proximal cannula portion (3), wherein the cannula (1) comprises a plurality of flexible filaments (4), such that the cannula (1) can be varied between a normal and a low-profile configuration, wherein the proximal cannula portion (3) is formed by a sleeve (5), in particular by a support sleeve (17), wherein the sleeve (5), in particular the support sleeve (17), comprises a, in particular external, thread (13) and/or a protrusion (12) and/or a slot (15) configured to establish a press fit and/or a twist and/or a clamp connection between the sleeve (5), in particular between the support sleeve (17), and a plug device (6).

15. Kit of parts comprising,
a cannula (1), in particular for use in medical applications, with a distal cannula portion (2) and a proximal cannula portion (3), wherein the cannula (1) comprises a plurality of flexible filaments (4), such that the cannula (1) can be varied between a normal and a low-profile configuration, wherein the proximal cannula portion (3) is formed by a sleeve (5), in particular by a support sleeve (17), and
a plug device (6) comprising a first (7) and a second (8) section, wherein the second section (8) is configured to be releasable positioned into the sleeve (5), in particular into the support sleeve (17), and the second section comprises an opening (9) to receive a proximal portion of an elongated member (10) and wherein the plug device (6) comprises at least one fastening member (11) configured to establish a press fit and/or a twist and/or a clamp connection between the sleeve (5), in particular the support sleeve (17), and the plug device (6), such that when the plug device (6) engages the sleeve (5), in particular the support sleeve (17), the cannula (1) is transferred from the normal to the low-profile configuration.
